(19) United States
(12) Patent Application Publication
(10) Pub. No.: US 2008/0213753 A1
(43) Pub. Date: Sep. 4, 2008

(54) METHOD FOR THE VERIFICATION OF THE REMOVAL OF VIRUSES TO VALIDATE FILTERS AND FILTERING PROCESSES

(75) Inventors: Hartmut HENNING, Goettingen (DE); Oscar-Werner Reif, Hannover (DE); Klaus Tarrach, Goettingen (DE); Robert Zeidler, Stahnsdorf (DE)

Correspondence Address: SUGHRUE MION, PLLC, 2100 PENNSYLVANIA AVENUE, N.W., SUITE 800, WASHINGTON, DC, 20037, US

(73) Assignee: SARTORIUS STEDIM BIOTECH GmbH, Goettingen (DE)

(21) Appl. No.: 12/047,470

(22) Filed: Mar. 13, 2008

Related U.S. Application Data
(63) Continuation of application No. PCT/EP2006/009002, filed on Sep. 15, 2006.

(30) Foreign Application Priority Data
Sep. 30, 2005 (DE) 102005047301.6

(51) Int. Cl. C12Q 1/70

(52) U.S. Cl. 435/5

(57) ABSTRACT

Disclosed is a method for verification of the removal of viruses to validate filters, filtering processes, physical and chemical inactivation processes, or adsorptive removal processes in predefined process conditions that are simulated on a small scale. According to said method, viruses are cultured in suitable cell lines in a first step, a virus suspension is obtained after solubilizing cells in a second step, the virus suspension obtained is added in a third step to a protein solution that is to be analyzed, and the virus-containing protein solution is filtered through the filter that is to be validated, and the removal of viruses is then analyzed in a fourth step. The virus suspension is first processed via a membrane adsorber following step two, the viruses being bonded to the membrane adsorber and contaminants being removed with the aid of a detergent buffer solution, while the bonded, purified viruses are eluted from the membrane adsorber area and are added to the protein solution that is to be analyzed as a concentrated virus suspension in step three.

This is a Continuation of International Application PCT/EP2006/009002, with an international filing date of Sep. 15, 2006, which was published under PCT Article 21(2) in German, and the complete disclosure of which is incorporated into this application by reference.

The invention relates to a method for verification of the removal of viruses for validation of filtration processes, physical and chemical inactivation methods or adsorptive removal methods under predefined process conditions that are simulated on a small scale, in which

in a first step, viruses are cultured in suitable cell lines,
in a second step, a virus suspension is obtained after cell digestion,
in a third step, the virus suspension obtained is added to a protein solution to be analyzed and
in a fourth step, the virus-containing protein solution is filtered through the filter that is to be validated and the removal of viruses is then analyzed.

According to the state of the art, verification of removal of viruses by filtration and other technologies is obtained in validation studies. Validation studies are usually conducted by qualified virus laboratory workers who work in accordance with the statutory requirements of GLP (Good Laboratory Practice). The basic procedure for the design and performance of validation studies is described in the CPMP document of the European authorities EMEA (CPMP: “Note for Guidance on Virus Validation Studies: The Design, Contribution and Interpretation of Studies Validating the Inactivation and Removal of Viruses,” February 1996 (CPMP/BWP/268/95)).

Validation in this context is understood to refer to a systematic review of the technologies selected for removal or inactivation of viruses, such as filtration, from the standpoint of the question of whether this technology is capable of removing or inactivating viruses under the stated process conditions of the user, which are to be simulated on a small scale (scaled down) in the virus laboratory to the extent recommended or even required on the part of the parties.

The specific requirements regarding the choice, use and expected removal rates (logarithmic removal rates, also referred to simply as LRR) of the viruses are depicted and explained in the ICH-Q5A document and the EMEA Guidelines (ICH-Q5A. “Viral Safety Evaluation of Biotechnology Products Derived from Cell Lines of Human or Animal Origin,” Federal Register, Vol. 63, No. 185, 1998) and (CPMP: “Note for Guidance on Virus Validation Studies: The Design, Contribution and Interpretation of Studies Validating the Inactivation and Removal of Viruses,” February 1996, CPMP/BWP/268/95).

Validation studies with the goal of determining the actual virus removal rate of certain technologies are also known as “spiking studies” because viruses are added to the product (spiked), usually a therapeutic protein solution as part of these studies, and then must be removed or inactivated to a certain extent by the respective technology that is to be validated.

The required total removal of all technologies used in the process is decided based on a risk analysis by the customer and a concluding assessment by the respective federal authority in which the approval and/or documents of the virus validation study are submitted. Total removal rates via a complete virus removal process (“virus clearance”) may be in a range from 12 log₁₀ to 24 log₁₀ increments and for individual technologies may be in a range from 3 log₁₀ to 7 log₁₀ increments.

The viruses are cultured by the responsible virus laboratories in suitable cell lines infected with the respective specific viruses and optionally obtained by digestion of the cells and from the cell culture supernatant. These cell cultures are specific for one or more viruses and are ordered by the virus laboratories from certain authorized sources.

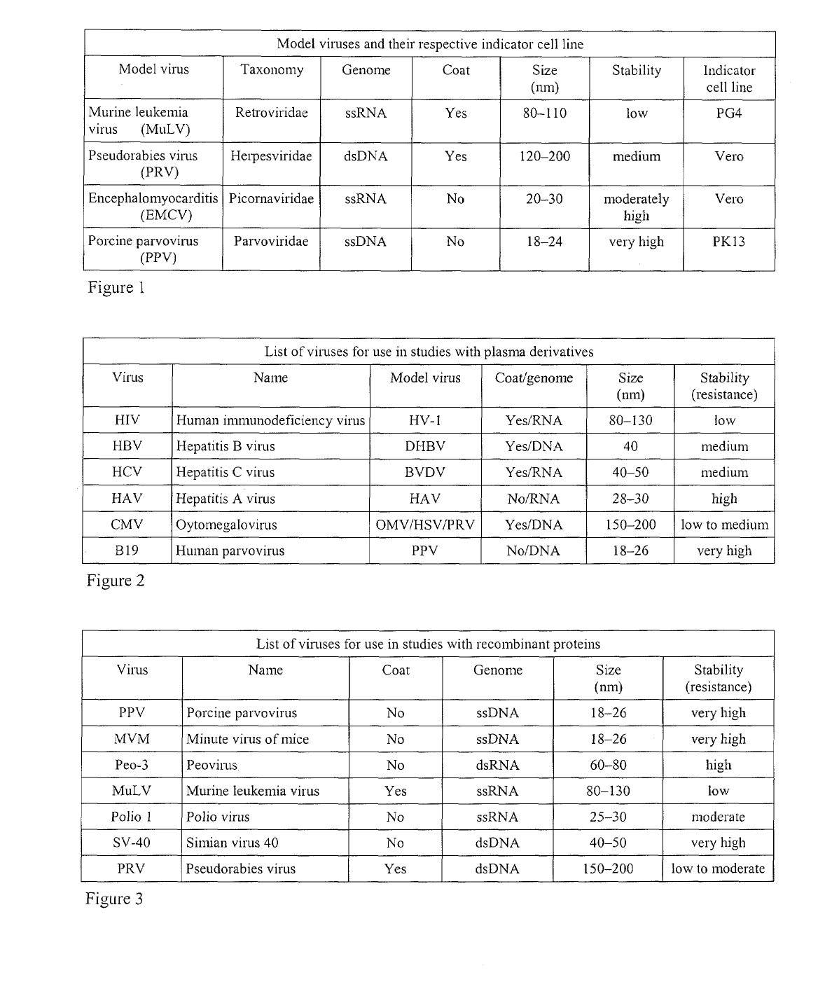

The table which has been included as FIG. 1 shows exemplary cell lines which are used for culturing viruses used in studies.

FIG. 2 shows a table with a list of viruses that are used for process validation of plasma derivatives.

FIG. 3 shows a table with a list of viruses for use in studies with recombinant proteins.

The virus concentration from a cell supernatant after culturing and cell digestion (e.g., by shock freezing and rethawing the solution) are in a range from 10⁶/mL to 10⁹/mL.

As part of spiking, these viruses suspensions are added to the corresponding protein solutions, but the addition of virus suspension is limited to max. 10 vol % (CPMP: “Note for Guidance on Virus Validation Studies: The Design, Contribution and Interpretation of Studies Validating the Inactivation and Removal of Viruses,” February 1996, CPMP/BWP/268/95).

After adding viruses to the product, virus titers of 10⁶/mL up to 10⁸/mL are achieved as a rule. This solution is then treated with the proper technology for virus removal or virus inactivation.

For filtration as the exemplary technology for virus removal (size exclusion principle, virus is retained based on its size, and the therapeutic protein solution flows through the membrane) certain volumes of virus-containing protein solution (the protein concentration of the solution is subject to the manufacturer's specifications and must be identical to the scale of the process; typical protein concentrations may be in the range from 10 μg/mL to 50 mg/mL solution) are filtered through the virus filter in these studies.

The amount of virus-containing solution to be filtered must be identical to the volume of the process filtration. This is achieved by scaling down the process quantities to the laboratory scale of the virus filter. Typical filtration quantities are in a range from 4 mL/cm² to 60 mL/cm² of virus filter area.

After filtration of the test solution through the filter to be validated, the virus removal is analyzed. The standard verification test is the TCID₅₀ infectivity test, which is described in the guidelines cited below:

G. Karber, Contribution to collective handling of pharmacological series experiments. Arch Exp Path Pharmkol 1931; 162:480-483.
Federal [German] Health Department and Paul Ehrlich Institute, Federal [German] Office for Sera and Vaccines (PEI). Announcement of the Approval of Pharmaceutical Drugs: Requirements of Validation Studies for Verification of Virus Safety of Drugs from Human Blood or Plasma, May 1994. Available from Bundesanzeiger [Federal Gazette], no. 84.

In these documents, both the test and the method of analysis are described. In addition to the TCID₅₀ test, there are ELISA tests and molecular biology methods (PCR, polymer chain reaction) for detection of viruses, if necessary.

One disadvantage of the known method is that addition of the virus supernatant, known as spiking, alters the product solution to be tested by introducing constituents into the protein solution which are not present on an actual process scale.

In addition to many biochemical substances from the spike, primarily host cell proteins and nucleic acids, cell fragments and culture aids as well as calf serum, all have disadvantages with regard to the validation study.

Another disadvantage for filtration or chromatographic purification, for example, within a validation study consists of the considerably inferior filterability of the virus-containing solution or problems in the chromatography process itself. Furthermore, the additional components introduced (nucleic acids, calf serum, salts) can influence the methods for quantification of viruses (e.g., ELISA and/or PCR). In addition, there are viruses which can be attracted only up to a certain concentration in the cell culture supernatant because of their natural situation. Then when up to 10% of such a supernatant is added within the context of the validation study, it regularly results in premature blockage of the filter, so the intended amount of volume per unit of area cannot be filtered. Then, as a direct consequence within the context of a virus validation study, the spike addition is reduced to the extent that the solution with the predefined volume can be filtered through the corresponding area of the virus filter.

In the extreme case, this may result in the dynamic range of possible virus removal (dynamic range=starting titer minus limit of detection) being no longer sufficient to be able to demonstrate at all the desired or required virus reduction of a certain technology. If this is the case, the only remaining alternative is to choose a larger filter area, which results in considerably higher filtration costs on a process scale.

The object of the present invention is therefore to provide a method which avoids the aforementioned disadvantages.

This object is achieved in conjunction with the preamble of claim 1 by the fact that after the second step, the virus suspension is first processed through a membrane adsorber; the viruses are bound to the membrane adsorber and unwanted constituents are removed with the help of a washing buffer, and the bound viruses are eluted from the membrane adsorber surface and in the third step are added as a purified concentrated virus suspension to the protein solution to be tested.

Work-up of the virus suspension, i.e., binding the viruses to a membrane adsorber with subsequent cleaning and elution of the viruses results in, for example, 100 mL virus supernatant being concentrated to a few mL eluate so that pure virus preparations in a higher concentration are available. Due to the fact that high starting titers in the spike solution and/or virus suspension are achieved, the volume per unit of area determined in the scale-down experiment, i.e., on the small scale of the user, may also be achieved in the virus validation study. The disadvantages mentioned above are thus safely and reliably avoided.

Additional preferred embodiment of the method is derived from the dependent claims.

According to a preferred embodiment of the invention, the membrane adsorber is a microporous anion or cation exchanger membrane with pore sizes>1 μm. Since diffusion limitation plays practically no role in membrane chromatography, it is possible to operate with relatively high flow rates.

According to a preferred embodiment of the inventive method, the infected cell lines are digested by a freezing process (shock freezing) with subsequent thawing and are centrifuged at approx. 2000 rpm over a period of approx. 10 min. The virus suspension obtained in this way is processed through a membrane adsorber with a microporous structure, such that the negatively charged viruses are bound to positively charged side chains of the membrane adsorber.

By using a washing buffer with an elevated salt concentration, less strongly binding contaminants can be removed. The pure virus can be then be eluted with a high molecular salt solution and rebuffered through a Vivaspin ultrafiltration unit, for example, and then is available in a highly concentrated form. The concentrated and purified virus suspension may then be added to the protein solution to be analyzed or stored temporarily. In a subsequent step the virus-containing protein solution is filtered in a known way through the filter that is to be validated and then the virus removal is analyzed.

### EXEMPLARY EMBODIMENT

To perform virus filtration, parvoviruses are replicated in a PK13 cell culture. The PK13 cells are obtained from the American Type Culture Collection, USA (ATCC No. CRL-2 6489). PK13 cell culture dishes, each with an area of 175 cm², are therefore infected with PPV, and three to five days later the viruses are harvested. The viruses are in the supernatant of the cells destroyed by infection and shock freezing. Approx. 40 mL supernatant is thus obtained from a culture flask. Forty-five flasks are used to prepare for virus filtration with a total of 1800 mL supernatant (obtained by centrifugation at 2000 rpm (approx. 800 g)±100 rpm at 10 min±1 min, after the cells are first destroyed completely by a freezing and thawing step). The supernatant was prefiltered with 0.45 μm of a membrane filter [sic; a 0.45 μm membrane filter] and then the titer was determined as 5.6*10⁸ /mL.

Of this, 300 mL are then used directly for virus filtration (suspension A). For work-up of the remaining 1500 mL (suspension B) with the membrane adsorber, the filtrate of the cell culture supernatant was loaded onto a membrane adsorber unit equilibrated with 50 mM Tris-HCl buffer, pH 7.5. The viruses then bind to ligands on the porous membrane structure.

By using a washing buffer (50 mM Tris-HCl, pH 7.5, 200-500 mM NaCl) with an elevated salt concentration, contaminants that bind less strongly can be removed. The purified virus itself is eluted with a high-molecular salt solution (50 mM Tris-HCl pH 7.5, 1.5M NaCl), rebuffered through a Vivaspin UF unit and then is available as an approximately 100× concentrated form in 16 mL buffer of choice or medium. The titer determined anew indicates a virus concentration of approx. 3.6×10¹⁰/mL. Virus suspension A produced by the traditional method as well as the new suspension B were both used for the virus filtration experiments.

To do so, a protein-containing solution (polyclonal antibody solution with 4 mg/mL in glycine buffer pH 4.1) was spiked with various concentrations of the virus suspensions A and B in different test runs and filtered through a 5 cm² virus filter (Saitorius Virosart CPV virus filter 20 nm nominal). Within the context of the experimental series, the filtration time, the maximum filtration volume with 75% blockage of the filter and the titer reduction achieved by the filter were determined.

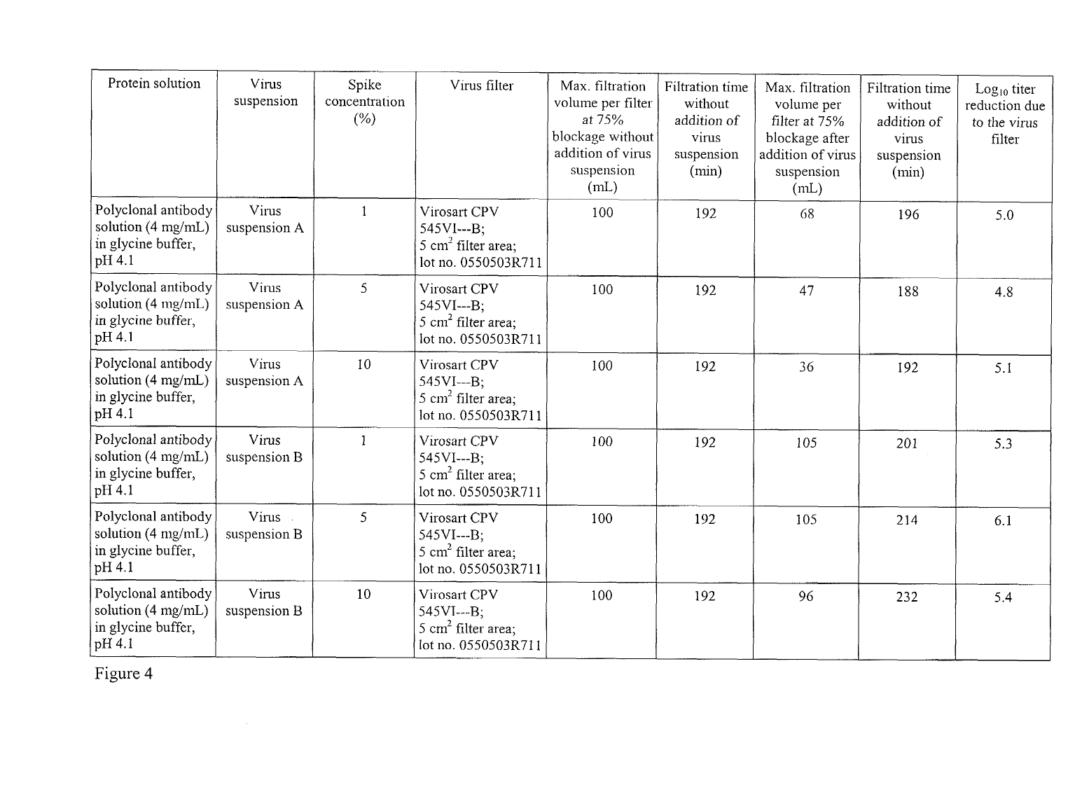

The results are summarized in FIG. 4 as the influence of virus work-up on filterability. This table shows clearly improved filterability of virus suspension B after purification by membrane chromatography. It is found that despite the high spike concentrations with virus suspension B (5% and more) the capacities of the virus filter are in a range that was found for the virus filter without the addition of a virus suspension. Thus the advantages described for the user in practice are manifested here to a particular extent.

The above description of the preferred embodiments has been given by way of example. These and other features of preferred embodiments of the invention are described in the claims as well as in the specification and the drawings. The individual features may be implemented either alone or in combination as embodiments of the invention, or may be implemented in other fields of application. Further, they may represent advantageous embodiments that are protectable in their own right, for which protection is claimed in the application as filed or for which protection will be claimed during pendency of this application or an application claiming benefit thereto. From the disclosure given, those skilled in the art will not only understand the present invention and its attendant advantages, but will also find apparent various changes and modifications to the structures and methods disclosed. The applicant seeks, therefore, to cover all such changes and modifications as fall within the spirit and scope of the invention, as defined by the appended claims, and equivalents thereof.

1. Method for verification of a virus reduction for validation of filters, filtration processes, physical and chemical inactivation methods or adsorptive removal methods under predefined process conditions which are simulated on a small scale, in which
in a first step, viruses are cultured in cell lines,
in a second step, a virus suspension is obtained after a cell digestion,
in a third step, the virus suspension thereby obtained is added to a protein solution to be analyzed and
in a fourth step, the virus-containing protein solution is filtered through the filter to be validated and then the virus removal is analyzed,
characterized in that after the second step, the virus suspension is processed first through a membrane absorber in which the viruses are bound to the membrane adsorber, contaminants are removed with the help of a washing buffer and the purified and bound viruses are eluted from the membrane absorber surface and in the third step are added as a concentrated virus suspension to the protein solution to be analyzed.

2. Method according to claim 1, characterized in that a microporous anion and cation exchanger membrane with pore sizes>1 μm is used as the membrane adsorber.

3. Method according to claim 1, characterized in that the infected cell lines are digested by a freezing process with subsequent thawing, and the virus suspension is obtained from a cell culture supernatant after prior centrifugation.

4. Method according to claim 3, characterized in that the centrifugation is performed at 2000 rpm±100 rpm over a period of 10 min±1 min.

5. Method according to claim 1, characterized in that the negatively charged viruses are bound to positively charged side chains of the membrane adsorber.

6. Method according to claim 5, characterized in that in the binding process the isoelectric point is adjusted between 3 and 6 and the pH is adjusted between 5.5 and 8.

7. Method according to claim 1, characterized in that the bound viruses are isolated with aqueous buffers with increasing salt content in the small volume from the membrane adsorber surface.
